(19) United States
(12) Patent Application Publication
(10) Pub. No.: US 2012/0190918 A1
(43) Pub. Date: Jul. 26, 2012

(54) APPARATUS AND METHODS FOR SUPPORTING CARDIAC ISCHEMIC TISSUE BY MEANS OF EMBEDDED STRUCTURES

(75) Inventors: Randolf Von Oepen, Los Altos, CA (US); Travis R. Yribarren, Coarsegold, CA (US); William E. Webler, JR., San Jose, CA (US); Barbara E. Stamberg, San Jose, CA (US); Florian N. Ludwig, Ebikon (CH); John J. Stankus, Campbell, CA (US); Yuet Mei Khong, Mannheim (DE)

(73) Assignee: ABBOTT CARDIOVASCULAR SYSTEMS, INC., Santa Clara, CA (US)

(21) Appl. No.: 13/011,850

(22) Filed: Jan. 21, 2011

(51) Int. Cl. A61F 2/04

(52) U.S. Cl. 600/37

(57) ABSTRACT

Systems and methods are disclosed for reinforcing ischemic tissue of a heart. A reinforcing element is initially positioned within a lumen of a delivery needle. The delivery needle is urged into the ischemic tissue and the reinforcing element is urged out of the needle into the ischemic tissue. The reinforcing element may be embodied as a coiled, undulating, or arcuate spring and may include a shape-memory material. A bioabsorbable material may maintain the reinforcing element in a deformed state. The reinforcing element may be tensioned as it is positioned within the myocardium in order to provide a cinching force by means of a cord lock selectively releasing the reinforcing element. The reinforcing element may be embodied as a number of spiral portions secured to a hub and urged outwardly by rotation of the hub.

### BACKGROUND OF THE INVENTION

1. The Field of the Invention

This application relates to apparatus and methods for treating ischemic heart disease.

2. The Relevant Technology

Congestive heart failure is a condition that results in the inability of the heart to fill or pump blood efficiently. Failure to treat congestive heart failure results in a gradual decline in heart function over time. Lifestyle changes such as an improved diet and increased physical activity can slow the progression of congestive heart failure. Certain drugs may also reduce the effects of the disease. However, the disease cannot presently be reversed. If untreated, congestive heart failure will ultimately require a complete heart transplant to prevent the death of the patient.

A specific manifestation of the disease is a weakening of the myocardium. As a result, the myocardium may become distended and sag. The weakened myocardium not only fails to contribute to the ability of the heart to pump blood, but also tends to expand during ventricular systole. The weakened region therefore causes a reduction of pressure within the ventricle and increases the volume of the ventricle at peak ventricular systole, thereby reducing the amount of blood flow.

### BRIEF SUMMARY OF THE INVENTION

These and other limitations may be overcome by embodiments of the present invention, which relates generally to medical devices and methods for treating ischemic heart disease.

In one aspect of the invention, an apparatus for supporting cardiac tissue including ischemic tissue includes a first anchor configured to engage the cardiac tissue and resist movement therethrough, a second anchor configured to engage the cardiac tissue and resist movement therethrough, and a biasing member engaging the first and second anchors and configured to urge the first anchor toward the second anchor.

In another aspect of the invention, the first and second anchors include barbs coupled to opposing ends of the biasing member.

In another aspect of the invention, the biasing element includes a first portion of a spring having a first outer diameter along a length thereof and the first and second anchors comprise second and third portions of the spring located on opposite sides of the first portion and having a second and third outer diameters, respectively, the second and third outer diameter being greater than the first outer diameter.

In another aspect of the invention, the biasing member includes a first arcuate member and a second arcuate member, a proximal end of the first arcuate member secured to a proximal end of the second arcuate member and the first anchor secured to a distal end of the first arcuate member and the second anchor secured to a distal end of the second arcuate member. The biasing member may further include a third arcuate member having a proximal end secured to the proximal ends of the first and second arcuate members. The distal end of the apparatus may further include a third anchor secure to the distal end of the third arcuate member. The first, second, and third arcuate members may include spirals each spiraling in the same direction. The first, second, and third arcuate members may include elastic wires and the first, second, and third anchors may include bent distal portions of the elastic wires.

In another aspect of the invention, the biasing element includes an elastic material having a relaxed shape. The apparatus may further include a bioabsorbable structure engaging the biasing element such that the biasing element is maintained in a deformed shape.

In another aspect of the invention, the biasing element includes a material that undergoes a change in shape responsive to an electric field.

In another aspect of the invention a method for treating cardiac tissue including ischemic tissue includes positioning a catheter adjacent the ischemic tissue and urging a reinforcing member out of a lumen of the catheter into the ischemic tissue.

In another aspect of the invention, the reinforcing member comprises a first anchor portion, a middle portion, and a second anchor portion and urging a reinforcing member out of the lumen includes urging the first anchor portion into the cardiac tissue, urging the middle portion into the cardiac tissue while applying tension to the middle portion, and urging the second anchor portion into the cardiac tissue while applying tension to the middle portion.

In another aspect of the invention, the method for treating cardiac tissue including ischemic tissue includes disengaging a locking mechanism from the second anchor portion. The second anchor portion may include an interference configured to engage the locking mechanism.

In another aspect of the invention, the method for treating cardiac tissue including ischemic tissue includes urging a delivery needle out of the lumen of the catheter into the ischemic tissue and urging the reinforcing member out of a lumen of the delivery needle. Urging the delivery needle out of the lumen of the catheter may include bending the delivery needle, such as by urging the needle out of an aperture defined by a lateral surface of the catheter and in communication with the lumen of the catheter.

In another aspect of the invention, a method for supporting ischemic tissue within a myocardium includes inserting a delivery needle within the myocardium, urging a reinforcing element outwardly from the delivery needle into the myocardium, the reinforcing element comprising a plurality of spiral portions each secured to a hub and having an anchor portion, and rotating the hub such that the spiral portions expand relative to the hub, the anchor portions of the spiral portions engaging the myocardium.

These and other advantages and features of the present invention will become more fully apparent from the following description and appended claims, or may be learned by the practice of the invention as set forth hereinafter.

### BRIEF DESCRIPTION OF THE DRAWINGS

To further clarify at least some of advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 is a schematic cross-sectional illustration of a heart having ischemic tissue;

FIG. 2 is a schematic isometric illustration of a heart having ischemic tissue adjacent a blood vessel;

FIG. 3A is an illustration of method for inserting a delivery needle for placement of a reinforcing element within the myocardium of a heart in accordance with an embodiment of the present invention;

FIG. 3B is an illustration of an alternative method for inserting a delivery needle for placement of a reinforcing element within the myocardium of a heart in accordance with an embodiment of the present invention;

FIG. 4 is an illustration of another alternative method for inserting a delivery needle for placement of a reinforcing element within the myocardium of a heart in accordance with an embodiment of the present invention;

FIGS. 5A and 5B are isometric views of embodiments of a reinforcing element in accordance with an embodiment of the present invention;

FIGS. 5C through 5F illustrate a method for placing a reinforcing element within the myocardium of a heart in accordance with an embodiment of the present invention;

FIGS. 6A and 6B are isometric views of alternative embodiments of a reinforcing element in accordance with an embodiment of the present invention;

FIGS. 6C through 6F illustrate a method for placing the reinforcing elements of FIGS. 6A and 6B within the myocardium of a heart in accordance with an embodiment of the present invention;

FIG. 7A illustrates another alternative embodiment of a reinforcing element in accordance with an embodiment of the present invention;

FIGS. 7B through 7E illustrate a method for placing the reinforcing element of FIG. 7A within the myocardium of a heart in accordance with an embodiment of the present invention;

FIGS. 8A through 8C illustrate a method for placing a reinforcing element embodied as a shape-memory element within the myocardium of a heart in accordance with an embodiment of the present invention;

FIGS. 9A and 9B illustrate a method for placing a reinforcing element including an electroactive material within the myocardium of a heart in accordance with an embodiment of the present invention;

FIG. 10 illustrates a reinforcing element including an electroactive material in accordance with an embodiment of the present invention;

FIGS. 11A through 11C illustrate a method for placing the reinforcing element of FIG. 10 within the myocardium of a heart in accordance with an embodiment of the present invention;

FIG. 12 illustrate a method for applying an electric potential to a reinforcing element including an electroactive material in accordance with an embodiment of the present invention;

FIGS. 13A through 13E illustrate reinforcing elements including a bioabsorbable material and methods for placing such reinforcing elements within the myocardium of a heart in accordance with an embodiment of the present invention;

FIGS. 14A and 14B illustrate a cord lock suitable for use in placing a reinforcing element within the myocardium of a heart in accordance with an embodiment of the present invention;

FIG. 15 is an isometric view of a locking pin suitable for use in a cord lock in accordance with an embodiment of the present invention;

FIGS. 16A and 16B illustrate a cord lock in locked and unlocked positions in accordance with an embodiment of the present invention;

FIGS. 17A through 17D illustrate a method for placing a reinforcing element using a cord lock in accordance with an embodiment of the present invention; and

FIGS. 18A through 18E illustrate a spiral shaped reinforcing element and methods for placing such a reinforcing element within the myocardium of a heart in accordance with an embodiment of the present invention.

### DETAILED DESCRIPTION OF THE PREFERRED EMBODIMENTS

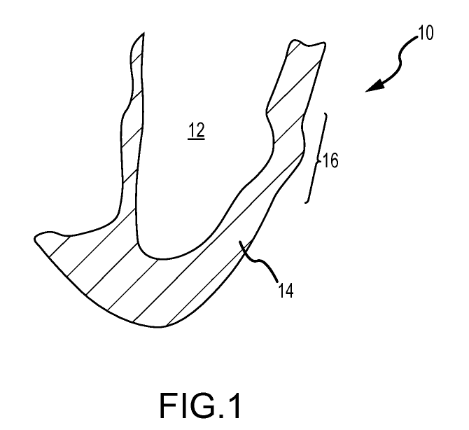

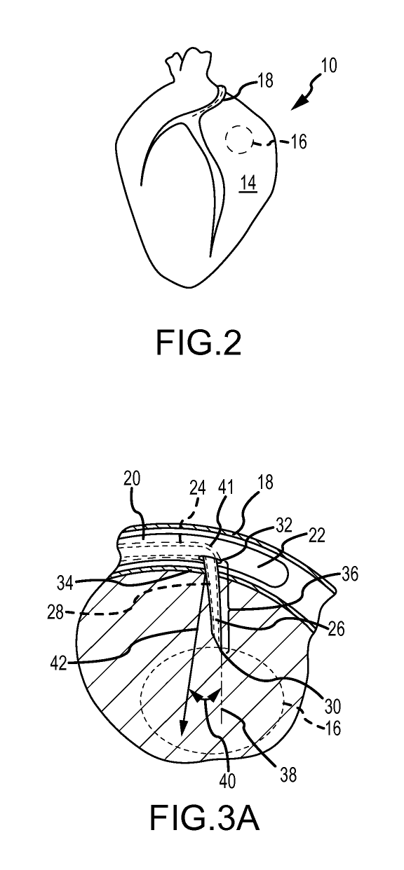

Referring to FIGS. 1 and 2, a heart 10 includes a ventricle 12 bounded by heart muscle or myocardium 14 that contracts to force blood out of the ventricle 12 during ventricular systole. A portion 16 of the myocardium 14 may have become ischemic, resulting in distension or sagging relative to the rest of the myocardium 14. Referring specifically to FIG. 2, on the surface of, and embedded in, the myocardium 14 are a number of blood vessels 18 supplying food and oxygen to the myocardium 14 and carrying away waste products and carbon dioxide. One or more of the vessels 18 may extend adjacent to the ischemic area 16.

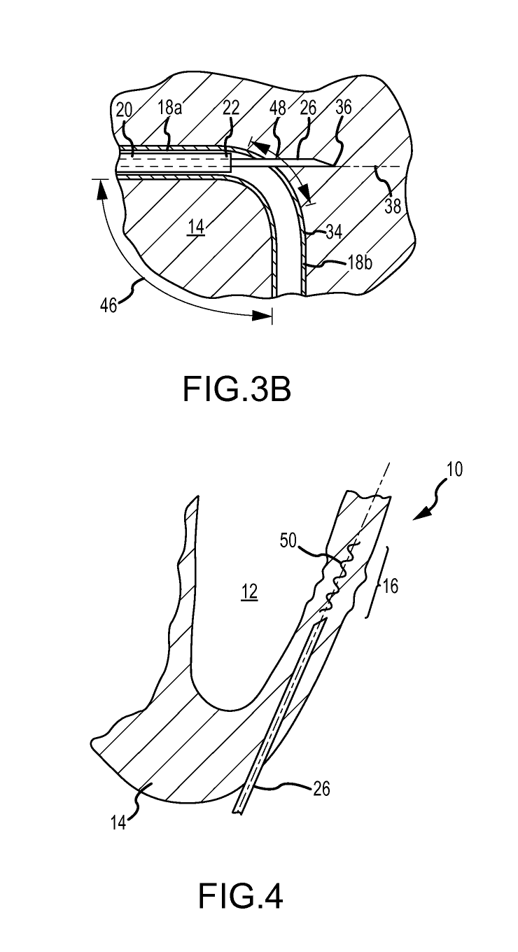

Referring to FIG. 3A, one or more reinforcing elements, described herein below, may be positioned within the ischemic area 16 percutaneously according to methods also described herein below. As shown in FIG. 3A, a catheter 20 may be threaded through the vasculature of a patient according to any suitable method known in the art such that the distal end 22 of the catheter 20 is located within the portion of the vessel 18 adjacent the ischemic area 16 and a proximal end is located external to a patient.

The catheter 20 defines a lumen 24 extending therethrough. A delivery needle 26 is positionable within the lumen 24. The needle 26 may define a lumen 28 extending therethrough and have a sharpened or beveled distal end 30 to facilitate penetration of vessel walls and the myocardium. The needle 26 may be formed of a biocompatible material having sufficient flexibility to enable threading through the catheter 20 that is itself threaded through a tortuous venous pathway. The needle 26 also preferably has sufficient rigidity to permit transfer of force from outside of the patient, through the catheter 20, to the distal end 30 of the needle 26 in order to selectively force the needle 26 out of the lumen 24 and into the myocardium 14. For example, a biocompatible semi-rigid polyester, polyamide or polyurethane polymer or a super-elastic metal such as nitinol may be suitable.

In use, the needle 26 is threaded through the lumen 24 of the catheter 24 to an aperture 32 in communication with the lumen 24. The needle 26 may be urged out of the aperture 32, through the vessel wall 34, and into the myocardium 14.

In the illustrated embodiment, the needle 26 is bent as it is urged outwardly from the lumen 24 such that a distal portion 36 of the needle 26 is oriented substantially normal to the vessel wall 34 at the point of penetration. For example, a longitudinal axis 38 of the distal portion 36 of the needle 26 may be at an acute angle 40 relative to a vector 42 normal to the vessel wall 34 at the point of penetration. For example, the angle 40 may be between 0 and 15 degrees.

The needle 26 may be bent by means of an arcuate portion 41 of the lumen 24 adjacent the aperture 32. In the illustrated embodiment, as the needle 26 is urged against the arcuate portion 41, the needle 26 bends and is forced out of the aperture 32. In the illustrated embodiment, the aperture 32 extends through the catheter 20 in a direction substantially perpendicular to the longitudinal axis of the catheter 20.

In an alternative embodiment, the catheter 20 may be actuated by means of tendons or other control means as known in the art. In such embodiments, the needle 26 may be bent as the catheter 20 is itself selectively bent in order to direct the needle 26 substantially perpendicular to the wall 34 of the vessel 18 at the point of penetration.

Referring to FIG. 3B, in some applications, a vessel 18 may have a bent shape including portion 18a, 18b that are at an angle 46 relative to one another. In such embodiments, the distal end 22 of the catheter 20 is positioned within portion 18a and the needle is urged outwardly from the lumen 24 and through a curved portion 48 of the vessel wall 34 extending between the portions 18a, 18b and across the longitudinal axis 38 of the distal portion 36 of the needle.

Referring to FIG. 4, in yet another alternative embodiment, a reinforcing element 50 may be urged outwardly from a needle 26 inserted into the myocardium 14 from the exterior of the ventricle 12 and vessels 18 of the heart 10. For example, either through an open chest or laparoscopic procedure, the needle 26 may be inserted through an outer surface of the myocardium 14 to the ischemic area 16 and the reinforcing element 50 urged outwardly from the needle 26.

Methods for introducing the reinforcing element 50 into the myocardium 14 from the needle 26 for reinforcement of the ischemic area 16 are illustrated below. The methods illustrated may be performed using a needle 26 positioned percutaneously or through an open chest or laparoscopic procedure.

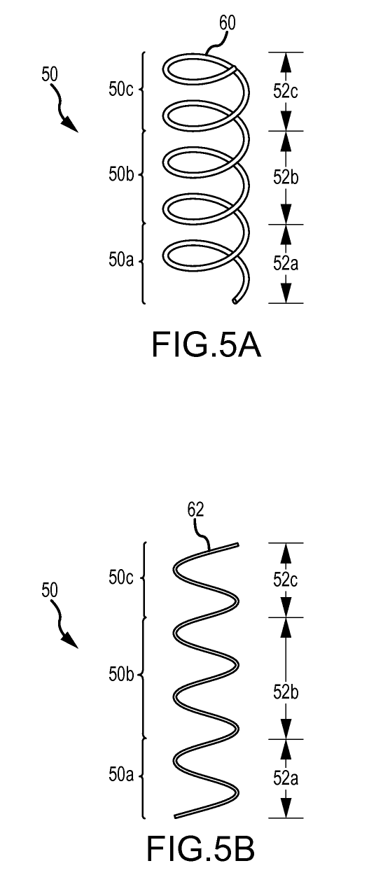

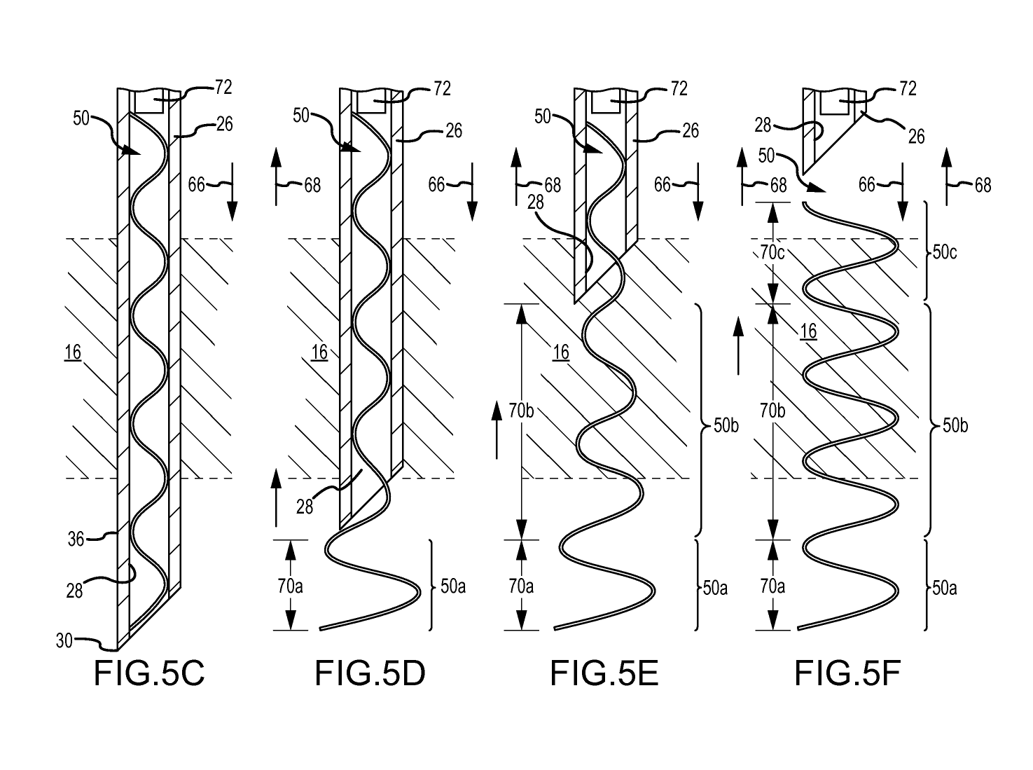

Referring to FIG. 5A, for purposes of illustrating the placement method, the reinforcing element 50 may be divided into a first anchor portion 50a, a middle portion 50b, and a second anchor portion 50c. In the embodiment of FIG. 5A, the reinforcing element 50 is embodied as a coil spring 60 and the different portions 50a-50c may simply be different portions of a coil spring having uniform coil diameter and pitch. Each of the portions 50a-50c has a corresponding length 52a-52c. Referring to FIG. 5B, in an alternative embodiment, the reinforcing element is embodied as spring 62 having a flat shape with an undulating or sinusoidal pattern within a plane. The springs 60, 62 may be formed of a resilient biocompatible polymer or metal. For example, the springs 60, 62 may be formed of nitinol. The surfaces of the springs 60, 62 may be textured or roughened to encourage tissue ingrowth in order to maintain the springs 60, 62 in position within the myocardium 14.

Referring to FIG. 5C, placement of the reinforcing element 50 embodied as either of the springs 60 or 62 within the myocardium 14 may include urging the needle 26 in a distal direction 66 through the myocardium either into or completely through the ischemic area 16 such that the distal end 30 is positioned either within the ischemic area 16 or within healthy tissue adjacent the ischemic area 16 with the needle 26 passing through the ischemic area 16. The reinforcing element 50 may be positioned within the distal portion 36 of the needle 26 as the needle 26 is urged into the myocardium or may be urged into the distal portion 36 after the needle 26 is urged into the illustrated position.

Referring to FIG. 5D, the first anchor portion 50a may be urged distally from the needle 26 along distal direction 66, such as by means of a push rod 72 extending from outside the patient, and through the lumen 28 of the delivery needle 26. The needle 26 may also be withdrawn in proximal direction 68 a distance 70a either before, after, or simultaneously with, urging of the first anchor portion 50a outwardly from the needle 26. The distance 70a may be substantially equal to, e.g., within 10 percent of, the relaxed length 52a of the first anchor portion 50a. The first anchor portion 50a may be positioned within a healthy tissue adjacent the ischemic area 16 or within the ischemic area 16.

Referring to FIG. 5E, the middle portion 50b may then be urged distally from the needle 26 along distal direction 66, such as by means of the push rod 72. The needle 26 may be withdrawn along proximal direction 68 a distance 70b either before, after, or simultaneously with, urging of the middle portion 50b distally from the needle 26. The distance 70b may be greater than the relaxed length 52b of the middle portion 50b such that the middle portion 50b is elastically deformed. For example, the distance 70b may be between 10 and 25 percent greater than the undeformed length 52b. The middle portion 50b preferably spans at least a portion of the ischemic area 16 in order to provide cinching and support to the ischemic area 16.

Referring to FIG. 5F, the second anchor portion 50c may then be urged distally from the needle 26 along distal direction 66, such as by means of the push rod 72. The needle 26 may be withdrawn a distance 70c either before, after, or simultaneously with, urging of the second anchor portion 50c outwardly from the needle 26. The distance 70c is preferably greater than the undeformed length 52c of the second anchor portion 50c such that the reinforcing element 50 completely exits the lumen 28. The second anchor portion 50c may be located within a healthy portion of the myocardium 14 adjacent the ischemic area 16 or within the ischemic area 16, or span both areas.

After the entire reinforcing element 50 has exited the lumen 28 the overall length of the reinforcing element 50 may reduce due to elastic restoring forces within the reinforcing element 50. However, the length of the reinforcing element 50 within the myocardium may be substantially greater than a relaxed length of the reinforcing element 50 such that the reinforcing element 50 continues to exert a biasing force on the myocardium 14 in order to provide a cinching and reinforcing force on the ischemic area 16.

As an alternative to the method described above with respect to FIGS. 5C through 5F, the reinforcing element 50 may simply be continuously urged distally from the lumen 28 of the needle 26 along distal direction 66 as the needle 26 is urged in proximal direction 68 in order to place the reinforcing element 50 within the ischemic area 16. The rate that the reinforcing element 50 is moved distally may be less than the rate at which the needle 26 is moved proximally such that the reinforcing element is elastically deformed.

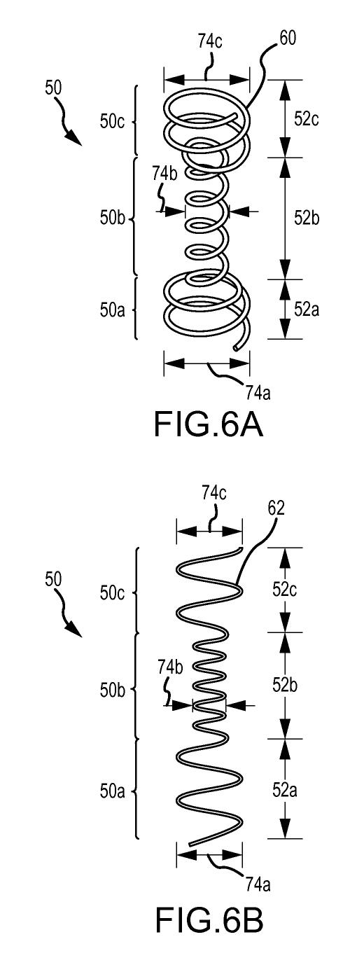

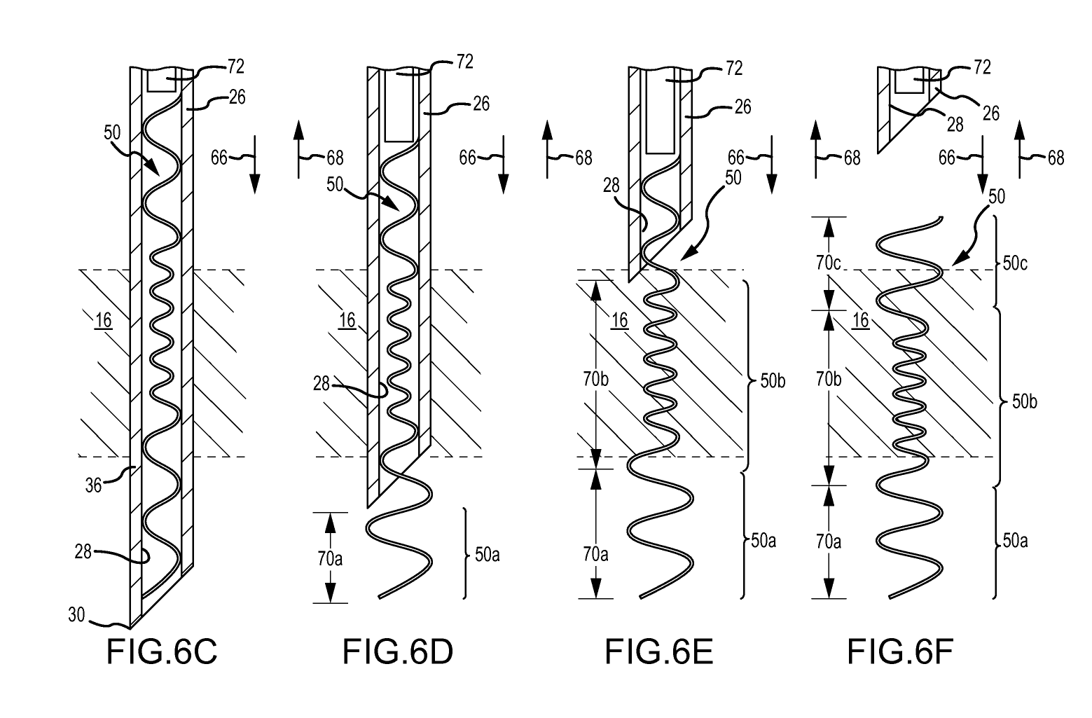

Referring to FIGS. 6A and 6B, in an alternative embodiment, the coil spring 60 or flat spring 62 include a first anchor portion 50a having a first width 74a, a middle portion 50b having a second width 74b, and a second anchor portion 50c having a third width 74c. The first width 74a and third width 74c are substantially larger than the second width 74b. For example, the first width 74a and third width 74c may be between 10 and 30 percent larger than the second width 74b. The first width 74a may be equal to or different from the third width 74c.

Referring to FIG. 6C, placement of the reinforcing element 50 embodied as either of the springs 60 or 62 within the myocardium 14 may include urging the needle 26 in a distal direction 66 through the myocardium either into or completely through the ischemic area 16 such that the distal end 30 is positioned either within the ischemic area 16 or within healthy tissue adjacent the ischemic area 16 with the needle 26 passing through the ischemic area 16. The reinforcing element 50 may be positioned within the distal portion 36 of the needle 26 as the needle 26 is urged into the myocardium 14 or may be urged into the distal portion 36 after the needle 26 is urged into the illustrated position.

Referring to FIG. 6D, the first anchor portion 50a may be urged distally from the needle 26 along distal direction 66, such as by means of a push rod 72 extending from outside the patient, and through the lumen 28 of the delivery needle 26. The needle 26 may also be withdrawn in proximal direction 68 a distance 70a either before, after, or simultaneously with, urging of the first anchor portion 50a outwardly from the needle 26. The distance 70a may be substantially equal to, e.g., within 15 percent of, the undeformed length 52a of the first anchor portion 50a. The first anchor portion 50a may be positioned within a healthy tissue adjacent the ischemic area 16 or within the ischemic area 16.

Referring to FIG. 6E, the middle portion 50b may then be urged distally from the needle 26 along distal direction 66, such as by means of the push rod 72. The needle 26 may be withdrawn along proximal direction 68 a distance 70b either before, after, or simultaneously with, urging of the middle portion 50b distally from the needle 26. The distance 70b may be greater than the undeformed length 52b of the middle portion 50b such that the middle portion 50b is elastically deformed. For example, the distance 70b may be between 10 and 30 percent greater than the undeformed length 52b. The middle portion 50b preferably spans the ischemic area 16 in order to provide cinching and support to the ischemic area 16. Inasmuch as the first anchor portion 50a has a greater width 74a than the width 74b the first anchor portion 50a will be better able to remain at its original placement position, rather than being dislodged as the middle portion 50b is tensioned.

Referring to FIG. 6F, the second anchor portion 50c may then be urged distally from the needle 26 along distal direction 66, such as by means of the push rod 72. The needle 26 may be withdrawn a distance 70c either before, after, or simultaneously with, urging of the second anchor portion 50c outwardly from the needle 26. The distance 70c is preferably greater than the undeformed length 52c of the second anchor portion 50c such that the reinforcing element 50 completely exits the lumen 28. The second anchor portion 50c may be located within a healthy portion of the myocardium 14 adjacent the ischemic area 16 or within the ischemic area 16.

After the entire reinforcing element 50 has exited the lumen 28 the overall length of the reinforcing element 50 may reduce due to elastic restoring forces within the reinforcing element 50. However, the length of the reinforcing element 50 within the myocardium may be substantially greater than an undeformed length of the reinforcing element 50 such that the reinforcing element 50 continues to exert a biasing force on the myocardium 14 in order to provide a cinching and reinforcing force on the ischemic area 16.

As with the first anchor portion 50a, because the second anchor portion 50c has a greater width 74c than the middle portion 50b, the second anchor portion 50c and first anchor portion 50a can support greater tension in the middle portion 50b without dislodging from their positions within the myocardium. The widened anchor portions 50a, 50c may be particularly useful where the anchor portions 50a, 50c are positioned within the ischemic area 16, rather than healthy tissue. Inasmuch as ischemic tissue is substantially weaker, the widened anchor portions 50a, 50c may sustain tension within the middle portion 50b without tearing the ischemic tissue of the ischemic area 16.

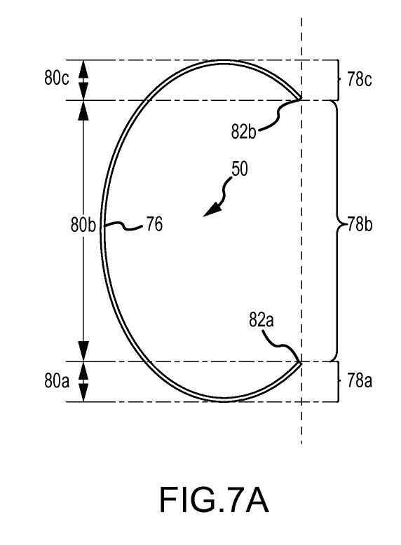

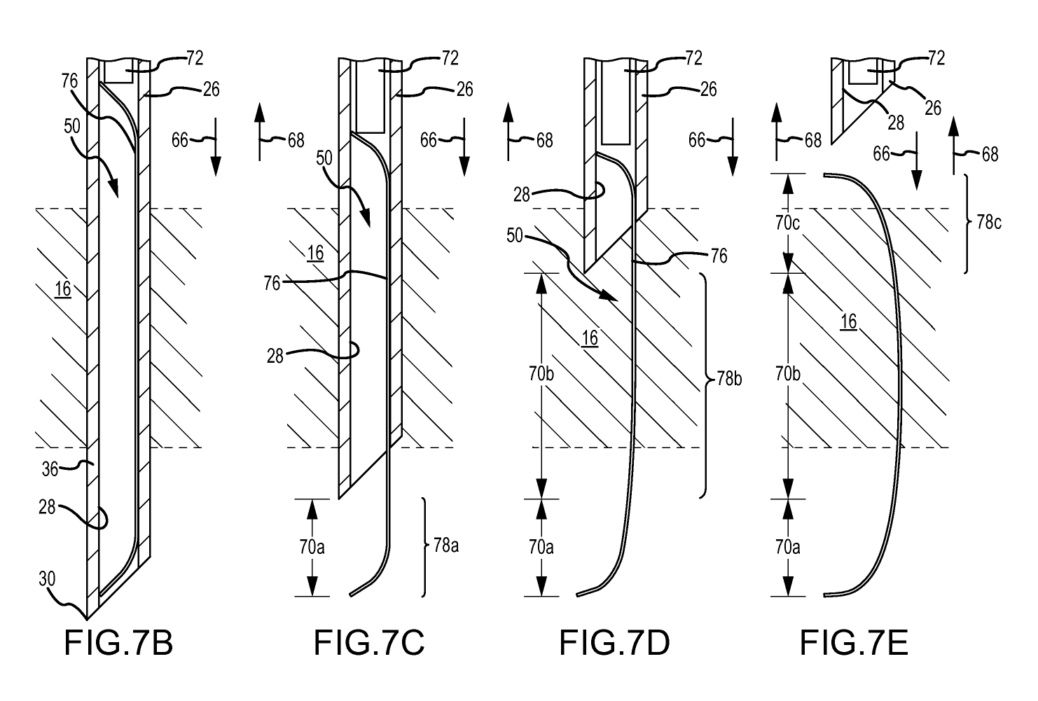

Referring to FIG. 7A, in an alternative embodiment, the reinforcing element 50 may be embodied as biasing element 76 having a curved shape of constant concavity. For example, the biasing element 76 may be substantially “C” shaped. The first anchor portion 50a and second anchor portion 50c may be embodied as first terminal portion 78a and second terminal portion 78c of the biasing element 76 and the middle portion 50b may be embodied as a middle portion 78b of the biasing element 76. The relaxed lengths 80a-80c of the first terminal portion 78a, middle portion 78b, and third terminal portion 78c, respectively, may be measured parallel to a line extending between the ends 82a, 82b of the biasing element 76.

The biasing element 76 may be formed of a resilient biocompatible polymer or metal. For example, the biasing element 76 may be formed of nitinol. The surfaces of the biasing element 76 may be textured or roughened to encourage tissue ingrowth in order to maintain the biasing element 76 in position within the myocardium 14.

Referring to FIG. 7B, placement of the reinforcing element 50 embodied as a biasing element 76 of constant concavity into the myocardium 14 may include urging the needle 26 in a distal direction 66 through the myocardium 14 either into or completely through the ischemic area 16 such that the distal end 30 is positioned either within the ischemic area 16 or within healthy tissue adjacent the ischemic area 16 with the needle 26 passing through the ischemic area 16. The biasing element 76 may be positioned within the distal portion 36 of the needle 26 as the needle 26 is urged into the myocardium or may be urged into the distal portion 36 after the needle 26 is urged into the illustrated position.

Referring to FIG. 7C, the first terminal portion 78a may be urged distally from the needle 26 along distal direction 66, such as by means of a push rod 72 extending from outside the patient and through the lumen 28 of the delivery needle 26. The needle 26 may also be withdrawn in proximal direction 68 a distance 70a either before, after, or simultaneously with, urging of the first terminal portion 78a outwardly from the needle 26. The distance 70a may be substantially equal to, e.g., within 10 percent of, the relaxed length 80a of the first terminal portion 78a. The first terminal portion 78a may be positioned within a healthy tissue adjacent the ischemic area 16 or within the ischemic area 16.

Referring to FIG. 7D, the middle portion 78b may then be urged distally from the needle 26 along distal direction 66, such as by means of the push rod 72. The needle 26 may be withdrawn along proximal direction 68 a distance 70b either before, after, or simultaneously with, urging of the middle portion 78b distally from the needle 26. The distance 70b may be greater than the relaxed length 80b of the middle portion 78b such that the middle portion 78b is elastically deformed. For example, the distance 70b may be between 10 and 30 percent greater than the relaxed length 80b. The middle portion 78b preferably spans the ischemic area 16 in order to provide cinching and support to the ischemic area 16.

Referring to FIG. 7E, the second terminal portion 78c may then be urged distally from the needle 26 along distal direction 66, such as by means of the push rod 72. The needle 26 may be withdrawn a distance 70c either before, after, or simultaneously with, urging of the second terminal portion 78c outwardly from the needle 26. The distance 70c is preferably greater than the undeformed length 80c of the second terminal portion 78c such that the biasing element 76 completely exits the lumen 28. The second terminal portion 78c may be located within a healthy portion of the myocardium 14 adjacent the ischemic area 16 or within the ischemic area 16.

After the entire biasing element 76 has exited the lumen 28 the overall length of the biasing element 76 may reduce due to elastic restoring forces within the biasing element 76. Where the reinforcing element 50 is embodied as biasing element 76 having constant concavity, the biasing element 76 may have an reduced average radius of curvature once the second anchor portion 50b is released from the needle 26 as compared to the average radius of curvature of the biasing element 76 when positioned entirely within the needle 26. However, the reduced average radius of curvature may still be substantially larger than the relaxed average radius of curvature of the relaxed biasing element 76 in the absence of any deforming forces.

As an alternative to the method described above with respect to FIGS. 7B through 7E, the reinforcing element 50 embodied as the biasing element 76 may simply be urged distally from the lumen 28 of the needle 26 as the needle 26 is withdrawn along proximal direction 68 in order to place the biasing element 76 within the ischemic area 16 without adhering to any predetermined relationship between the rate of exit from the lumen 28 and a rate of withdrawal of the needle 26. Alternatively, the rate that the biasing element 76 is urged outwardly may be less than the rate at which the needle 26 is withdrawn such that the biasing element 76 is elastically deformed.

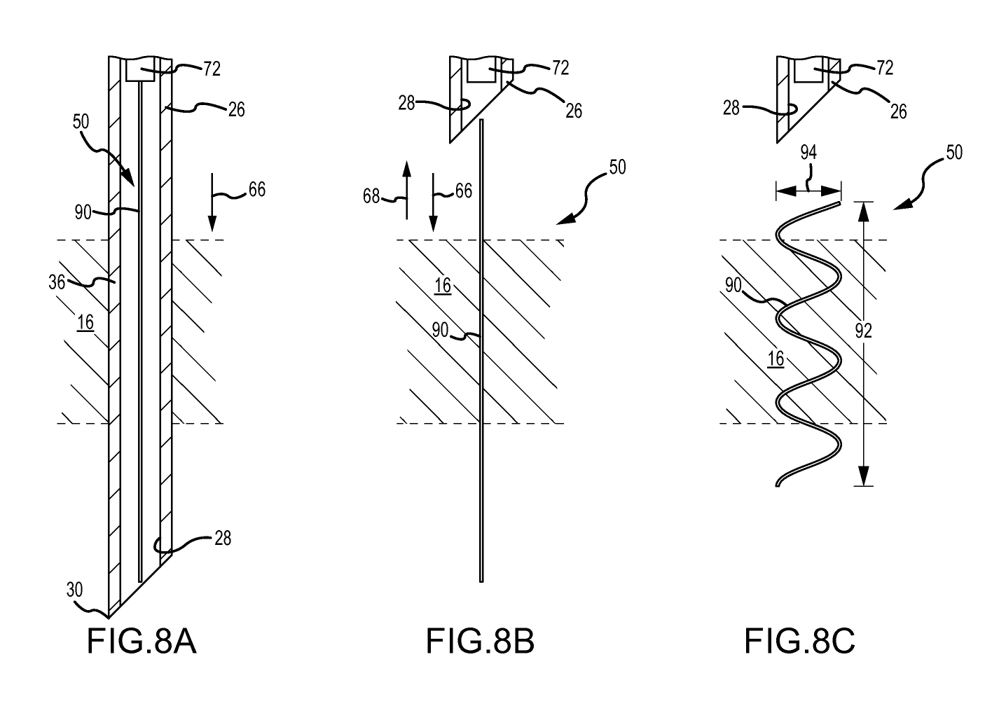

Referring to FIGS. 8A-8C, in some embodiments, the reinforcing element 50 may be embodied as a shape-memory element 90. For example, the shape-memory element 90 may include a shaped memory material (“SMM”) or superelastic material. For example, the SMM can be shaped in a manner that allows for restriction to induce a substantially linear orientation while within the lumen 28 of the needle 26, but can automatically retain an undulating memory shape shown in FIG. 8C. SMMs have a shape memory effect in which they can be made to remember a particular shape. Once a shape has been remembered, the SMM may be bent out of shape or deformed and then returned to its original shape by unloading from, strain, heating, or other environmental stimuli such as light. SMMs can be shape memory alloys (“SMA”) or superelastic metals comprised of metal alloys, or shape memory plastics (“SMP”) comprised of polymers.

A SMA can have any non-characteristic initial shape that can then be configured into a memory shape by heating the SMA and conforming the SMA into the desired memory shape. After the SMA is cooled, the desired memory shape can be retained. This allows for the SMA to be bent, straightened, compacted, and placed into various contortions by the application of requisite forces; however, after the forces are released, the SMA can be capable of returning to the memory shape. The main types of SMAs are as follows: copper-zinc-aluminium; copper-aluminium-nickel; nickel-titanium (“NiTi”) alloys known as nitinol; and cobalt-chromium-nickel alloys or cobalt-chromium-nickel-molybdenum alloys known as elgiloy. The nitinol and elgiloy alloys can be more expensive, but have superior mechanical characteristics in comparison with the copper-based SMAs. The temperatures at which the SMA changes its crystallographic structure are characteristic of the alloy, and can be tuned by varying the elemental ratios.

For example, the primary material of the shape-memory element 90 can be of a NiTi alloy that forms superelastic nitinol. Nitinol materials can be trained to remember a certain shape, straightened in a shaft, catheter, or other tube, and then released from the catheter or tube to return to its trained shape. Also, additional materials can be added to the nitinol depending on the desired characteristic.

An SMP is a shape-memory plastic that can be fashioned into the shape-memory element 90 in accordance with the present invention. When a SMP encounters a temperature above a glass transition or melting point in a polymer, the polymer makes a transition to a more rubbery or fluid state. The elastic modulus can change more than two orders of magnitude across a thermal transition temperature (“Tₜᵣ”). When this transition temperature, Tₜᵣ, is less than the highest transition temperature in a copolymer it can be used to impose a temporary shape. As such, an SMP can be formed into a desired shape of the shape-memory element 90 by heating it above the Tₜᵣ, fixing the SMP into the new shape, and cooling the material below Tₜᵣ. The SMP can then be arranged into a temporary shape by force and then resume the memory shape once the force has been applied. Examples of SMPs include, but are not limited to, biodegradable polymers, such as poly(ε-caprolactone) dimethacrylate, multiblock copolyesters from poly(ε-caprolactone) and PEG, multiblock copolymers with poly(L-lactide) and poly(D,L-lactide-co-ε-caprolactone) or poly(glycolide-co-ε-caprolactone), polyesterurethanes based on poly(ε-caprolactone) soft segments, polyetheresters, and non-biodegradable polymers such as, polynorborene, polyisoprene, styrene butadiene, non-degradable polyurethane-based materials, vinyl acetate-polyester-based compounds, poly(ethylene-co-vinyl acetate) and others yet to be determined. As such, any SMP can be used in accordance with the present invention.

In addition, shape memory light sensitive polymers may also be employed. For example acrylate based polymers that are end-capped with cinnamic acid, cinnamylidene acetic acid, or other suitable material can be strained from an original shape and crosslinked via UV light with a wavelength greater than about 260 nm to hold a desired temporary shape. Upon irradiation at with UV light with a wavelength of less than 260 mm, the previously-formed crosslinks are cleaved and the material can return to its original shape. In addition, illumination of the light sensitive polymer could be effectuated by the introduction of light through a light fiber positioned within push rod 72. Alternatively, the light fiber could replace push rod 72 and serve the same function.

Also, it can be beneficial to include at least one layer of an SMA and at least one layer of an SMP to form a multilayered body; however, any appropriate combination of materials can be used to form a multilayered medical device.

The shape-memory element 90 can be comprised of a variety of known suitable deformable materials, including stainless steel, silver, platinum, tantalum, palladium, cobalt-chromium alloys such as L605, MP35N, or MP20N, niobium, iridium, any equivalents thereof, alloys thereof, and combinations thereof. The alloy L605 is understood to be a trade name for an alloy available from UTI Corporation of Collegeville, Pa., including about 53% cobalt, 20% chromium and 10% nickel. The alloys MP35N and MP20N are understood to be trade names for alloys of cobalt, nickel, chromium and molybdenum available from Standard Press Steel Co., Jenkintown, Pa. More particularly, MP35N generally includes about 35% cobalt, 35% nickel, 20% chromium, and 10% molybdenum, and MP20N generally includes about 50% cobalt, 20% nickel, 20% chromium and 10% molybdenum.

Also, the shape-memory element 90 can include a suitable biocompatible polymer in addition to or in place of a suitable metal. The polymeric shape-memory element 90 can include a biocompatible material, such as biostable, biodegradable, or bioabsorbable materials, which can be either plastically deformable or capable of being set in the deployed configuration. If plastically deformable, the material can be selected to allow the medical device to be expanded in a similar manner using an expandable member so as to have sufficient radial strength and scaffolding and also to minimize recoil once expanded. If the polymer is to be set in the deployed configuration, the expandable member can be provided with a heat source, light source, or infusion ports to provide the required catalyst to set or cure the polymer. Biocompatible polymers are well known in the art, and examples are recited with respect to the polymeric matrix. Thus, shape-memory element 90 can be prepared from a biocompatible polymer.

Referring specifically to FIG. 8A, placement of the reinforcing element 50 embodied as a shape-memory element 90 within the myocardium 14 may include urging the needle 26 in a distal direction 66 through the myocardium 14 either into or completely through the ischemic area 16 such that the distal end 30 is positioned either within the ischemic area 16 or within healthy tissue adjacent the ischemic area 16 with the needle 26 passing through the ischemic area 16. The reinforcing element 50 may be positioned within the distal portion 36 of the needle 26 as the needle 26 is urged into the myocardium or may be urged into the distal portion 36 after the needle 26 is urged into the illustrated position.

Referring to FIG. 8B, the shape-memory element 90 may then be urged distally from the needle 26 along distal direction 66. The needle 26 may also be withdrawn along proximal direction 68 as the shape-memory element 90 is urged outwardly from the needle 26, such as by means of a push rod 72. In an alternative embodiment, the needle may be positioned adjacent the ischemic area such that the long slender shape-memory element 90 may then be urged out of the needle 26 and penetrate through the ischemic area 16.

Referring to FIG. 8C, following introduction into the ischemic area 16, the shape-memory element 90 may transition to a memory shape a different from an initial shape possessed by the shape-memory element 90 within the needle 26. For example, the shape-memory element 90 may transition from a substantially straight member to a member having an undulating pattern or a coil shape, such as the reinforcing elements 50 illustrated in FIGS. 4A through 6F. Alternatively, the shape-memory element 90 may transition from a straight member to a member having a curved shape of constant concavity such as the biasing element 76 of FIG. 7A. In some embodiments, the initial shape of the shape-memory element 90 includes undulations or coils that have a larger pitch than that of the memory shape of the shape-memory element 90.

Transitioning of the shape-memory element 90 from the original shape of FIG. 8A to the memory shape of FIG. 8C may result in shortening of the length 92 of the shape-memory element 90 and an increase in the width 94 thereof such that the shape-memory element 90 exerts a biasing force on surrounding ischemic tissue within the ischemic area resulting in reinforcement and cinching of the ischemic tissue.

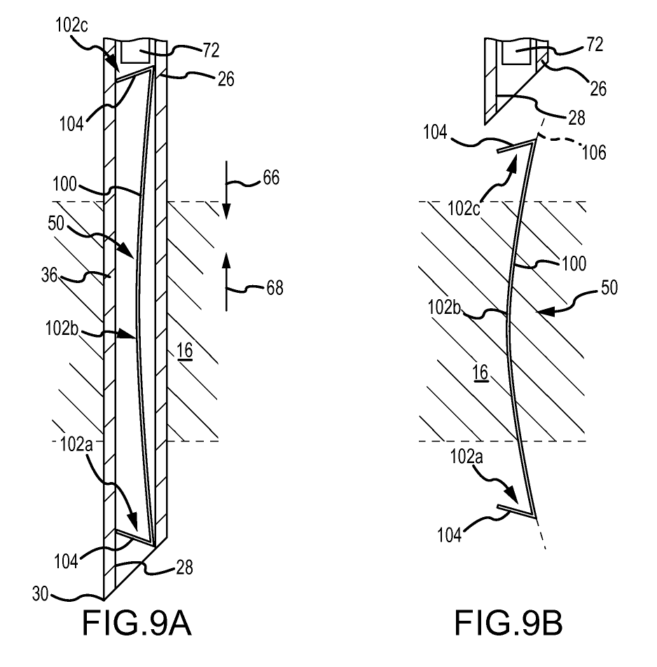

Referring to FIGS. 9A and 9B, in an alternative embodiment, the reinforcing element 50 is embodied as an electro-actuated element 100. The electro-actuated element 100 may include a material that undergoes shortening in response to an applied electric potential. The shortening may be permanent or may relax in the absence of an applied field. For example, the electro-actuated element 100 may include a piezoelectric material or an electroactive polymer such as an artificial muscle. Electroactive materials include conductive polymers such as polypyrrole and polyaniline that are doped with surfactants such as sodium dodecyl benzene sulfonate. Polythiophenes doped with a surfactant such as sodium dodecyl benzene sulfonate are also suitable. Other electroactive materials include derivatives of polyacetylene, poly(phenylene sulfide), poly(p-phenylene vinylene)s, poly(3,4-ethylenedioxythiopene), polyethylenedioxythiophene, Poly(vinylidene fluoride) or PVDF and its copolymers, poly(vinylidene fluoride-trifluoro-ethylene) copolymer, Nafion® (perfluorosulphonate manufactured by Du Pont), or Flemion® (perfluorocaboxylate manufactured by Asahi Glass, Japan) impregnated with conductive metals such as gold and platinum and carbon nanotubes. Another example of electroactive polymers are ionic polymer metal composites such as perfluorsulfonate polymers that include small amounts of sulfonic or carboxyic ionic functional groups. Other varieties of electroactive polymers exist, and so this explanation is not intended to be exhaustive but could be expanded upon by one skilled in the art.

In the illustrated embodiment, the electro-actuated element 100 includes a first anchor portion 102a, a middle portion 102b, and a second anchor portion 102c. In some embodiments, only the middle portion 102b includes an electro-actuated material whereas the first and second anchor portions 102a, 102b are formed of static material such as a resilient biocompatible polymer or a biocompatible metal such as nitinol. In the illustrated embodiment the first and second anchor portions 102a, 102b are embodied as hooked or barbed portions 104 formed on either side of the middle portion 102b. In an alternative embodiment, the first and second anchor portions 102a, 102c may be embodied as coiled portions, undulating portions, or another structure extending transversely from the longitudinal axis 106 of the middle portion 102b.

As in the other embodiments described hereinabove, placement of the electro-actuated element 100 may include urging the needle 26 in a distal direction 66 through the myocardium 14 either into or completely through the ischemic area 16 such that the distal end 30 is positioned either within the ischemic area 16 or within healthy tissue adjacent the ischemic area 16 with the needle 26 passing through the ischemic area 16. The electro-actuated element 100 may be positioned within the distal portion 36 of the needle 26 as the needle 26 is urged into the myocardium or may be urged into the distal portion 36 after the needle 26 is urged into the illustrated position. The electro-actuated element 100 may then be urged in distal direction 66 as the needle 26 is withdrawn in the proximal direction 68 until the electro-actuated element 100 exits the lumen 28 of the needle 26 as shown in 9B. An electric potential may then be applied to the electro-actuated element 100 in order to cause shortening thereof, resulting in cinching and supporting of the ischemic tissue within the ischemic area 16.

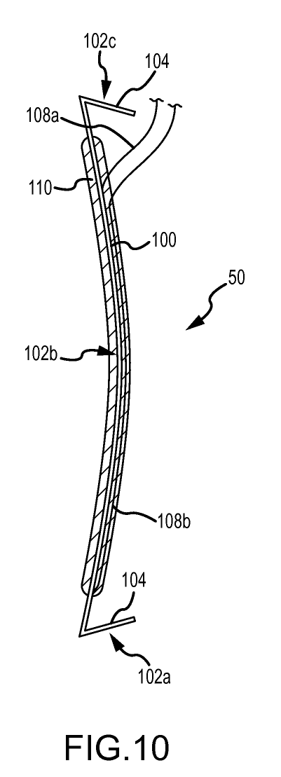

Referring to FIG. 10, in one embodiment, two or more conductive leads 108a, 108b secure to different points on the middle portion 102b of the electro-actuated element 100. Insulative material 110 may coat the conductive leads 108a, 108b to prevent shorting.

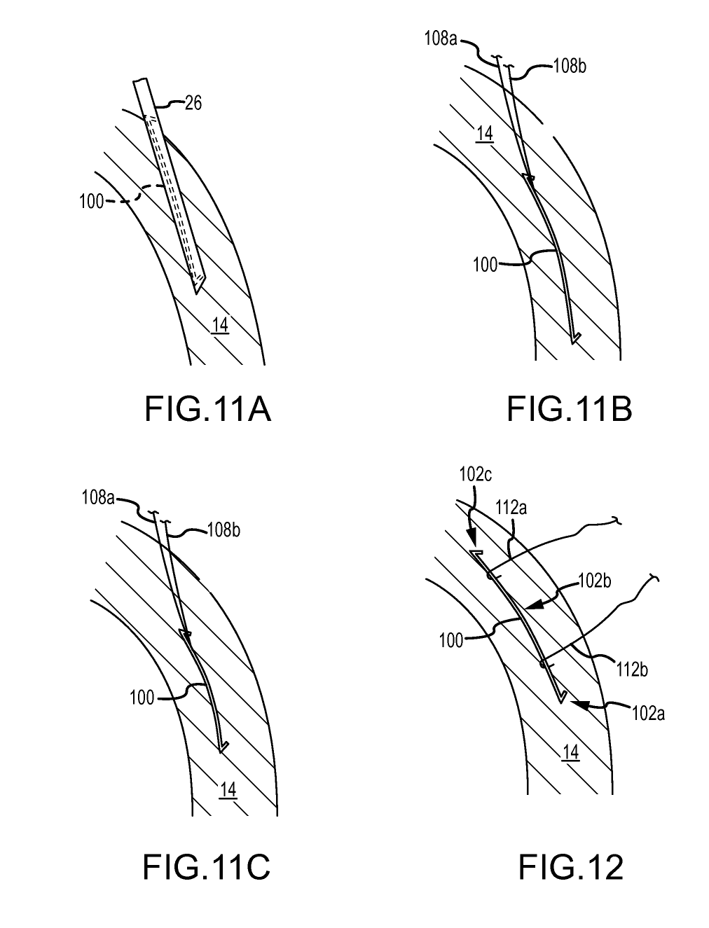

Referring to FIGS. 11A and 11B, the conductive leads 108a, 108b may be coupled to a source of electric power by inserting the needle 26 from outside of the heart into the myocardium 14. As the needle 26 is withdrawn and the electro-actuated element 100 is urged into the myocardium, the conductive leads 108a, 108b may also exit the needle 26 such that portions of the leads 108a, 108b protrude outside of the myocardium 14. An electric potential may be imposed on the leads 108a, 108b in order to cause shortening of the electro-actuated element 100 as shown in FIG. 11C. The portions of the leads 108a, 108b protruding from the myocardium may then be removed or may be left in place for future use.

Referring to FIG. 12, in an alternative embodiment, following placement of the electro-actuated element 100 within the myocardium 14, probes 112a, 112b may be inserted through the myocardium 14 to contact the middle portion 102b of the electro-actuated element 100. An electric potential may then be applied to the probes 112a, 112b to cause shortening of the electro-actuated element 100.

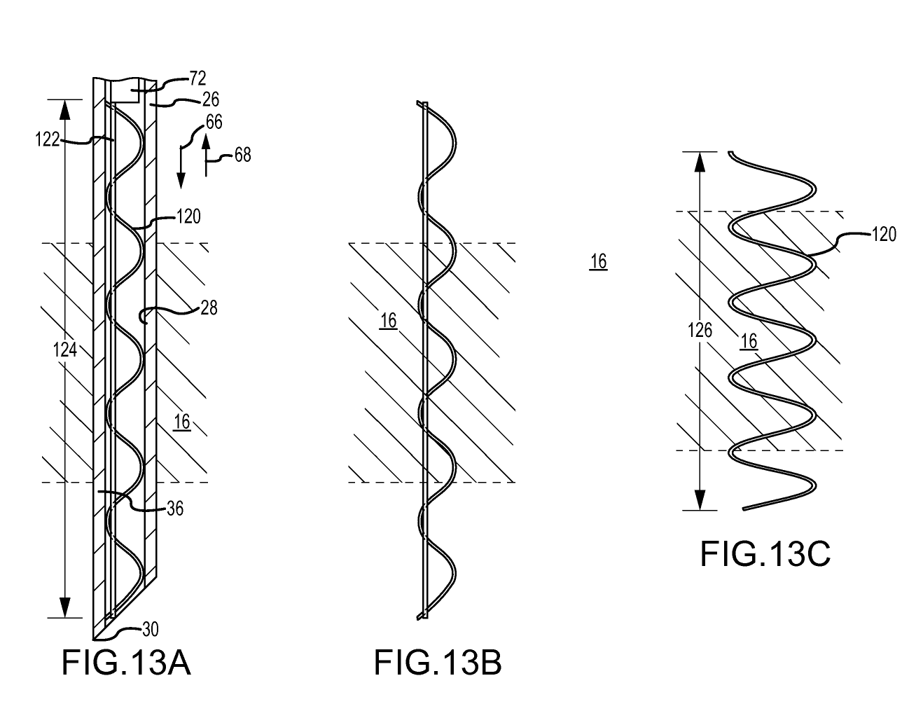

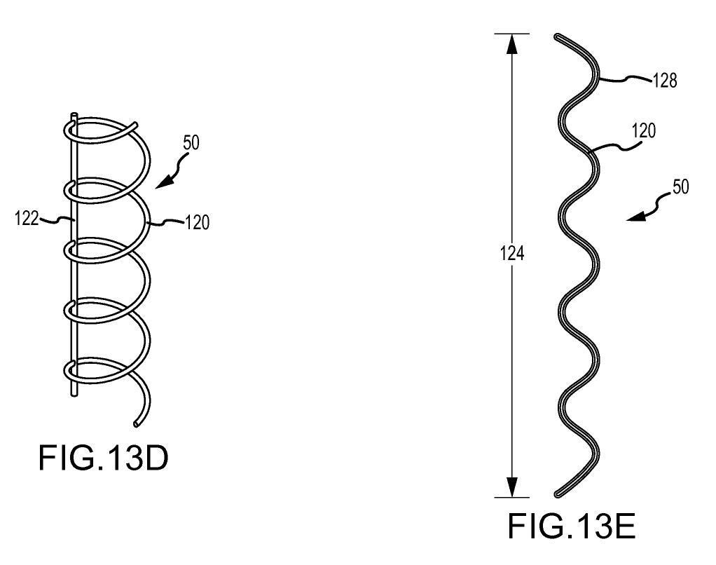

Referring to FIGS. 13A and 13B, in some embodiments the reinforcing element 50 may be embodied as a biasing element 120 coupled to a bioabsorbable element 122. The biasing element 120 has a deformed length 124 while positioned within the needle 26 that is longer than a relaxed or undeformed length of the biasing element 120. The bioabsorbable element 122 engages the biasing element 120 and maintains the biasing element 120 in a deformed shape. In the illustrated embodiment, the biasing element 120 has an undulating shape and the bioabsorbable element 122 engages the undulations at two or more points to prevent contraction of the biasing element 120.

As in the other embodiments described hereinabove, placement of the biasing element 120 may include urging the needle 26 in a distal direction 66 through the myocardium 14 either into or completely through the ischemic area 16 such that the distal end 30 is positioned either within the ischemic area 16 or within healthy tissue adjacent the ischemic area 16 with the needle 26 passing through the ischemic area 16. The biasing element 120 may be positioned within the distal portion 36 of the needle 26 as the needle 26 is urged into the myocardium or may be urged into the distal portion 36 after the needle 26 is urged into the illustrated position. The biasing element 120 may then be urged in distal direction 66 as the needle 26 is withdrawn in the proximal direction 68 until the biasing element 120 exits the lumen 28 of the needle 26 as shown in 13B.

Referring to FIG. 13C, over time, the bioabsorbable element 120 will erode and the biasing element 120 will recoil toward an undeformed or relaxed shape such that it has a length 126 substantially less than the deformed length 124. For example, the length 126 of the biasing element 120 within the myocardium 14 may be between 5 and 20 percent less than the deformed length 124. The presence of the myocardium 14 may inhibit complete relaxation of the biasing element 120.

The biasing element 120 and bioabsorbable element 122 may have various configurations. Referring to FIG. 13D, in an alternative embodiment, the biasing element 120 is shaped as a coil or helical spring having the bioabsorbable element 122 engaging separate loops of the coil or helix. Alternatively, the biasing element 120 may have an undulating pattern or a coil shape, such as the reinforcing elements 50 illustrated in FIGS. 4A through 6F. Alternatively, the biasing element 120 may have a curved shape of constant concavity such as the biasing element 76 of FIG. 7A.

Referring to FIG. 13E, in some embodiments, the bioabsorbable element 122 may be embodied as a coating 12 including a bioabsorbable material applied to the biasing element 120 while stretched to the deformed length 124. Following curing of the coating 128 the coating 128 may have sufficient rigidity to maintain the biasing element 120 in a deformed state.

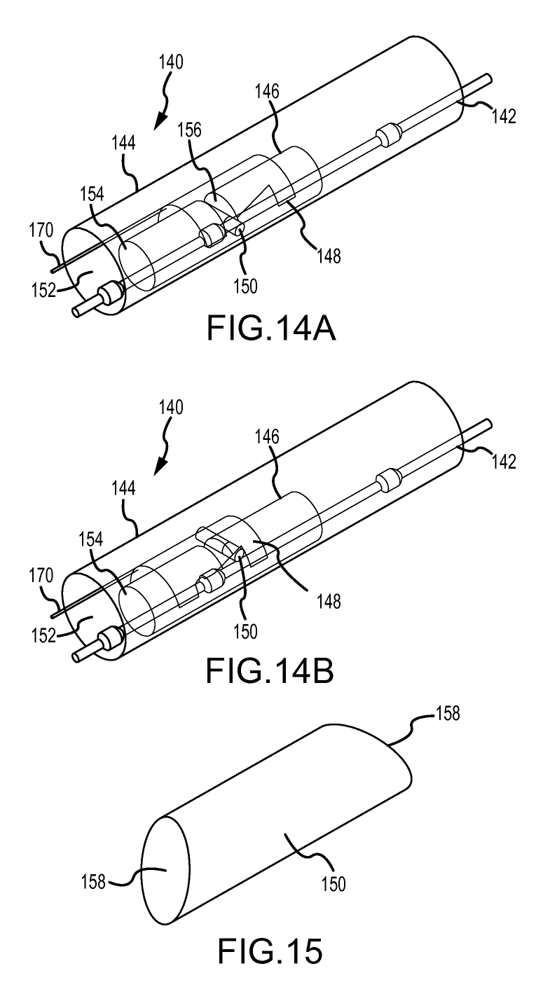

Referring to FIGS. 14A and 14B, a reinforcing element 50 may be tensioned using a cord lock mechanism described in U.S. patent application Ser. No. 10/740,360, filed Dec. 17, 2003. FIGS. 14A and 14B are three-dimensional partial see-through views of the locking device 140 with the cord 142 in an unlocked condition (FIG. 14A) and the cord 142 in a locked condition (FIG. 14B). For purposes of the present application the cord 142 may be embodied as a proximal portion of a reinforcing element 50.

In some embodiments, the locking device 140 includes an outer housing 144 and inner housing 146 having tubular shapes as shown in FIGS. 14 and 14B. The tubular shape makes the design, construction and manufacturing of the outer housing 144, the locking member 148 and the inner housing 146 easier and more compatible with catheter and cord constructions. In some embodiments, the outer housing 144 and the inner housing 146 may have other configurations such as square, oval, hexagonal, etc. The outer housing 144 may extend through the lumen 28 of the needle 26 to a point located outside of a patient and function as a push rod for positioning of the reinforcing element 50 and the locking device 140 by an operator. Alternatively, the needle 26 may function as the outer housing and a separate outer housing 144 may be omitted. In such embodiments, a push rod, or like structure, may secure to the inner housing 146 and extend to a position outside of a patient for positioning the locking mechanism 140 within the lumen 28 of the needle 26.

In one embodiment, the locking device 140 further includes a locking member 148, and a locking pin 150. In one embodiment, the inner housing 146, the locking member 148, and the locking pin 150 form the locking mechanism of the locking device 140. The locking mechanism is configured so that it can lock the cord 142 in position relative to the locking device 140. The locking mechanism is also configured so that it can unlock the cord 142 from a locked condition.

In one embodiment, the outer housing 144 has a lumen 152 extending longitudinally therethrough. The cord 142 is disposed within the lumen 140. The cord 142 is freely moveable through the lumen 152 except when it is in a locked condition. The cord 142 is further disposed in a lumen 154 of the inner housing 146. The inner housing 146 is disposed within the lumen 152 of the outer housing 144.

In one embodiment, the inner housing 146 is attached to one side of the outer housing 144. Alternatively, the inner housing 146 may be secured to a separate push rod for positioning of the chord lock 140 and the outer housing 144 may be omitted. In such embodiments, the inner housing 146 is freely movable within the lumen 28 of the needle 26. In another embodiment, inner housing 146 is mechanically constrained from free longitudinal and rotational movement relative to outer housing 144. For example, features such as holes, slots, tabs and tangs can be included on the outer housing 144 and the inner housing 146 that engage or cooperate with one another to mechanically constrain the inner housing 146 from longitudinal and/or rotational movement relative to the outer housing 140.

The locking member 148 is disposed within the lumen 152 and over a portion of the outer surface of the inner housing 146. The locking member 148 is configured to be able to move longitudinally over the cord 142 and place the cord 142 in a locked or unlocked condition. In one embodiment, the locking member 148 is configured to be able to move longitudinally on the outer surface of the inner housing 146 and place the cord 142 in a locked or unlocked condition.

In one embodiment, the inner housing 146 is provided with an opening 156 extending laterally, or transversely, through a portion of the inner housing 146. The opening 156 cooperates with the locking member 148 to allow the locking member 148 to lock the cord 142 into position or unlock the cord 142 from a locked condition. The locking member 148 is disposed on the outer surface of the inner housing 146 and configured to lock or unlock the cord 142 through the opening 140.

In one embodiment, the locking pin 150 is disposed within the opening 156. The locking pin 150, the opening 156, and the locking member 148 work together to lock or unlock the cord 140. To lock or unlock the cord 142, the locking member 148 is configured to move longitudinally over the inner housing 146 (over the outer surface of the inner housing 146). The moving of the locking member 148 allows the locking pin 150 to move up in opening 156, e.g. transversely to the longitudinal axis of the inner housing 146, thereby releasing the cord 142 or causes the locking pin 150 to be held down within opening 156, constraining the cord 140. The cord 142 can be placed in a locked condition when the locking pin 150 is held down by the position of locking member 148 and the position of the locking pin 150 restricts the longitudinal motion of cord 140. The cord 142 can be placed in an unlocked condition when the position of locking member 148 is such that locking pin 150 is free to move up, releasing the cord 142 and allowing the cord 142 to move longitudinally freely through the lumen 154 of the inner housing 140.

In one embodiment, the opening 156 created into a portion of the inner housing 146 exposes the cord 142. The opening 156 is perpendicular to the longitudinal axis of the inner housing 146. The locking pin 150 sits in the opening 156. The locking pin 150 is configured so that it does not lodge into the lumen 154 of the inner housing 146. In one embodiment, the locking pin 150 is configured to be longer than the outer diameter or the width of the inner housing 146. The locking pin 150 is also configured to be shorter than the inner diameter of the outer housing 144 so that its movement is partially constrained by the internal walls of the outer housing 144, which may be embodied as the inner walls of the lumen 28 of the needle 26. In such configurations, the locking pin 150 is prevented from lodging into the inner diameter of the inner housing 146.

In one embodiment, the locking pin 150 resides in the opening 156 in a way such that it can engage portions of the locking member 148 and keep the locking member 148 from longitudinally sliding off the outer surface of the inner housing 146 in either direction. In other embodiments, the inner housing 146, the outer housing 144 and/or the device of which the locking device 140 is a part of, attaches to or otherwise communicates with, includes features (e.g., tangs, tabs or other mechanical projections) that constrain the longitudinal motion of the locking member 148 in a single direction or in both directions. The locking pin 150 is shown as having a circular cross-section and a rod shape in FIGS. 14A and 14B, but, of course, in other embodiments, the locking pin 150 may have many other cross-sections and shapes without deviating from the scope of the embodiments of the present invention. A circular cross-section helps reduce forces required to operate the locking device 140 (see later portions of this description for the significance of the forces). A rod shape is the simplest shape to manufacture, as it may be easily formed or cut to the desired length from many materials widely available as preformed wire or rod using common processes.

Referring to FIG. 15, in some embodiment, the locking pin 150 is configured to include beveled (pointed) ends 158. In this embodiment, instead of having straight cut ends, the locking pin 150 includes the beveled ends 158 to provide the locking pin 150 with the greatest movement range within the lumen 152.

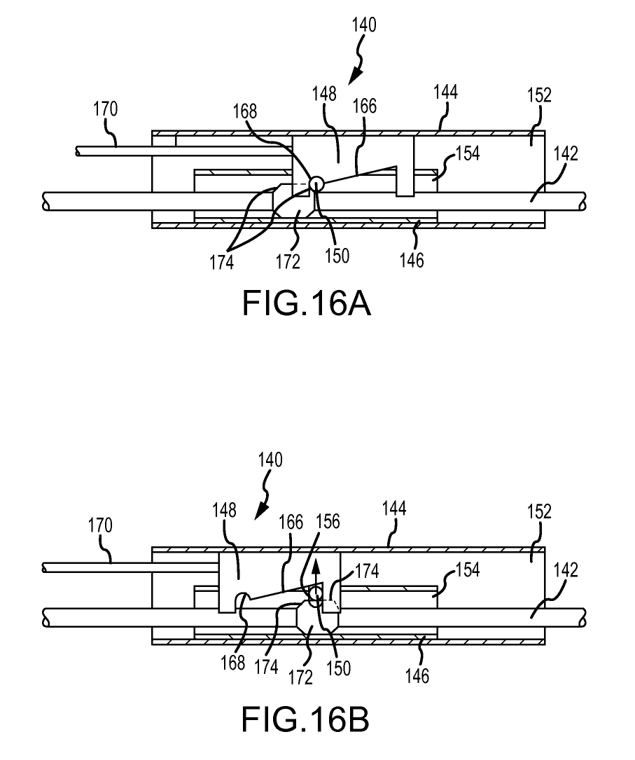

As shown in FIGS. 16A and 16B, the locking pin 150 needs to be able to move up and down within the opening 156 and relative to the inner diameter of the outer housing 144 (and inner housing 146) to cooperate with the locking member 148 to lock or unlock the cord 140. When the locking pin 150 is configured with straight cut ends, the locking pin 150 would hit the inner wall of the outer housing 144 and be limited as to its up and down travel distance or range. With the beveled ends 158, the locking pin 150 can move up and down with a greater travel distance. In addition, with the beveled ends, the inner diameter (ID) of outer housing 144 (hence, the outer diameter (OD) of locking device 140) can be configured to have a smaller dimension than would otherwise be the case when the locking pin 150 has straight cut ends. In other locking pin 150 cross-sections, such as those where the interaction of the locking pin 150 with the opening 156 prevents the locking pin 150 from rotating (e.g., a square, rectangular or oval cross-section), a bevel(s), an incline(s) or a curve(s) may be placed on the appropriate side(s) of the end(s) of the locking pin 150 to facilitate a greater travel distance in the up and/or down direction(s).

In one embodiment, as shown in FIGS. 16A and 16B, the locking member 148 is disposed on the outer surface of the inner housing 146 partially filling at least a portion of the gap between the inner surface of the outer housing 144 and the outer surface of inner housing 140. The locking member 148 is slightly smaller than the dimensions of the gap between the inner surface of the outer housing 144 and the outer surface of inner housing 146, so that the locking member 148 may slide freely longitudinally within this gap. In addition, the locking member 148 is captured from moving longitudinally off of the outer surface of the inner housing 146 and out of the inside of the locking device 140 by its engagement with the protrusions of the locking pin 150 on either side of the inner housing 146.

The locking member 148 includes an incline 166 and an indent 168 on each side of the locking member 140. The indent or detent 168 is configured so that it can engage the locking pin 150 to limit the motion of the locking pin 150, as shown in FIG. 16A, such that the locking pin 150 is held near or against the bottom of opening 156 of the inner housing 146 and/or in contact with or close to the ID of the outer housing 140. The incline 166 is configured such that at its upper limit, it may constrain the locking pin 150 from moving up and out of the opening 156 and/or allow the locking pin 150 to be constrained by its contact with the ID of the outer housing 140.

The locking member 148 may be actuated by means of a tether 170 coupled thereto. Tension applied to the tether 170 moves the locking member 148 relative to the locking pin 150, such that the locking pin 150 is forced out of the indent 168, as shown in FIG. 16B, and allowed to move adjacent the incline 166, as shown in FIG. 16C. As the locking member 148 is moved further toward the unlocked position the locking pin 150 is able to travel up the incline 166.

As the locking pin travels up the incline 166, the locking pin disengages from the cord 142. In one embodiment, the locking pin is disengaged from interferences 172 formed on the cord 142. The interference 172 can be bumps created on the outer surface of the cord 140. In this embodiment, the cord 142 can be composed of or coated with a low friction and relatively stiff material such as Nylon, Polyethylene (PE), Polytetrafluoroethylene (PTFE) or Polyetheretherketone (PEEK), or any number of biocompatible polymers. In one embodiment, an interference 172 is created by shrink melting sections of a miscible material into the coating of or directly into the cord 140.

In one embodiment, shrink melting sections of nylon tubes onto the nylon coating of the cord 142 or the cord 142's coating creates the interferences 172. In another embodiment, tubes or other shapes with a compatible inner diameter are placed over the outer diameter of the cord 142 and crushed, welded, soldered, brazed, glued or crimped in place to form the interferences 172 on the outside of the cord 140. In another embodiment, the interferences 172 are molded onto the surface of the cord 142 or a coating of the cord 142. In one embodiment, the interferences 172 have curved, inclined or beveled ends 174 to aid in the smooth movement of the cord 142 through the locking device 140. These type of ends 174 on the interference 172 also aid in providing forces that help retain the locking pin 150 in the indent or detent 168 (and thus help assure that the locking device 140 will remain locked) when an interference 170 is forced up against the locking pin 150 in the locked condition by the forces acting on the cord 140.

In one embodiment, in the locked condition, the interferences 172 will cause a mechanical interference with the locking pin 150 to the section of the cord 142 that has the interferences 172 such that, when the locking pin 150 engages the interferences 172, the cord 142 is locked into a position between the interferences 170 or against an interference 172 and the cord 142 is not allowed to freely move longitudinally in at least one direction within the inner housing 146.

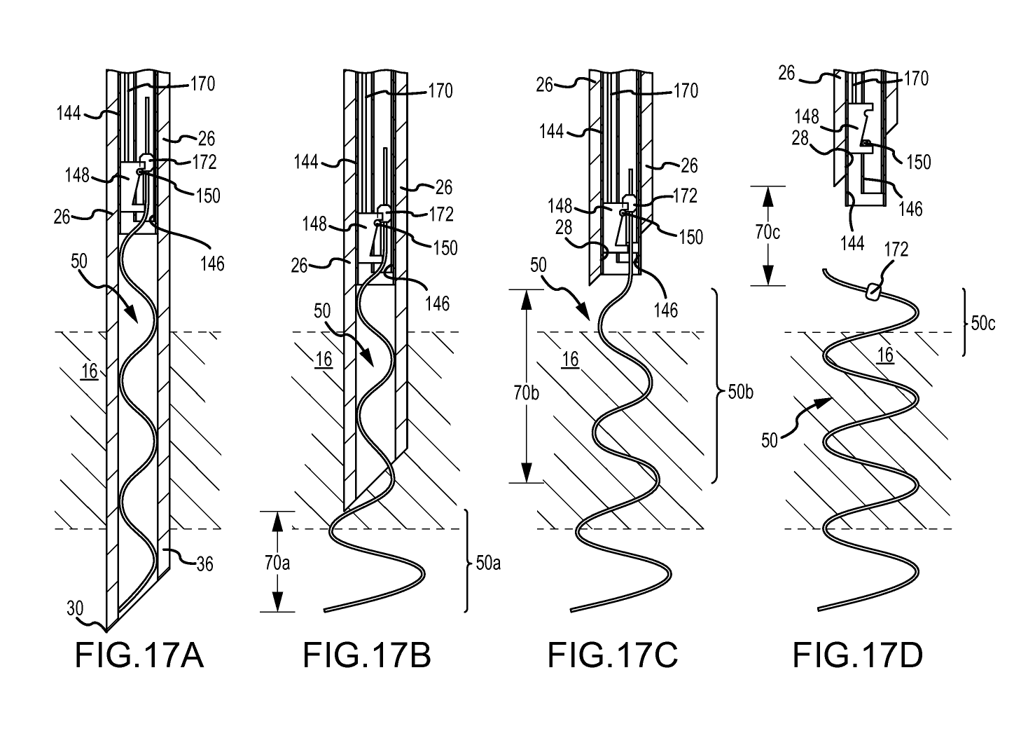

Referring to FIG. 17A, a reinforcing element 50 suitable for use with the cord locking mechanism 140 may include an interference 172 formed on a proximal portion 180 thereof. The reinforcing element 50 may be embodied as any of the reinforcing elements described hereinabove, such as the coil spring 60 or flat spring 62 of FIGS. 5A through 5F and FIGS. 6A through 6F or the arcuate biasing element 76 of FIGS. 7A through 7E.

The interference 172 may be positioned within the locking mechanism 140 in a locked condition having the reinforcing element 50 positioned within the lumen 28 of the needle 26. As in the other embodiments described hereinabove, the needle 26 may be urged either into or completely through the ischemic area 16. The reinforcing element 50 may be positioned within the distal portion of the lumen 28 either during or after insertion of the needle 26.

Referring to FIG. 17B, the first anchor portion 50a may be urged distally from the needle 26 along distal direction 66, such as by means of the outer housing 144 or a push rod secured to the inner housing 146. Either the outer housing 144 or push rod may extend from outside the patient, and through the lumen 28 of the delivery needle 26. The needle 26 may also be withdrawn in proximal direction 68 a distance 70a either before, after, or simultaneously with, urging of the first anchor portion 50a outwardly from the needle 26. The distance 70a may be substantially equal to, e.g., within 10 percent of, the undeformed length 52a of the first anchor portion 50a. The first anchor portion 50a may be positioned within a healthy tissue adjacent the ischemic area 16 or within the ischemic area 16.

Referring to FIG. 17C, the middle portion 50b may then be urged distally from the needle 26 along distal direction 66, such as by means of pressure applied to the outer housing 144 or a push rod secured to the inner housing 146. The needle 26 may be withdrawn along proximal direction 68 a distance 70b either before, after, or simultaneously with, urging of the middle portion 50b distally from the needle 26. The distance 70b may be greater than the undeformed length 52b of the middle portion 50b such that the middle portion 50b is elastically deformed. For example, the distance 70b may be between 10 and 30 percent greater than the undeformed length 52b. The middle portion 50b preferably spans the ischemic area 16 in order to provide cinching and support to the ischemic area 16.

The cord lock 140 may advantageously enable the distal portion 180 of the reinforcing element 50 to be firmly retained within the lumen 28 during the step illustrated in FIG. 17C and thereby enable substantial tensioning of the middle portion 50b.

Referring to FIG. 17D, the second anchor portion 50c may then be urged distally from the needle 26 along distal direction 66, such as by means of pressure applied to the outer housing 144 or a push rod secured to the inner housing 146. The needle 26 may be withdrawn a distance 70c either before, after, or simultaneously with, urging of the second anchor portion 50c outwardly from the needle 26. The distance 70c is preferably greater than the undeformed length 52c of the second anchor portion 50c such that the reinforcing element 50 completely exits the lumen 28. The second anchor portion 50c may be located within a healthy portion of the myocardium 14 adjacent the ischemic area 16 or within the ischemic area 16.

Before, simultaneous with, or after, urging of the second anchor portion 50c outwardly from the lumen 28 of the needle 26, the cord lock 140 may be transitioned to an unlocked condition, thereby allowing the distal portion and interference 172 to exit the cord lock 140. This may be accomplished by applying tension in the proximal direction 68 on the tether 170 to urge the indent 168 of the locking member 148 out of engagement with the locking pin 150, such that the pin is able to slide upwardly along the incline 166 within the opening 156.

After the entire reinforcing element 50 has exited the lumen 28 the overall length of the reinforcing element 50 may reduce due to elastic restoring forces within the reinforcing element 50. However, the length of the reinforcing element 50 within the myocardium may be substantially greater than an undeformed length of the reinforcing element 50 such that the reinforcing element 50 continues to exert a biasing force on the myocardium 14 in order to provide a cinching and reinforcing force on the ischemic area 16.

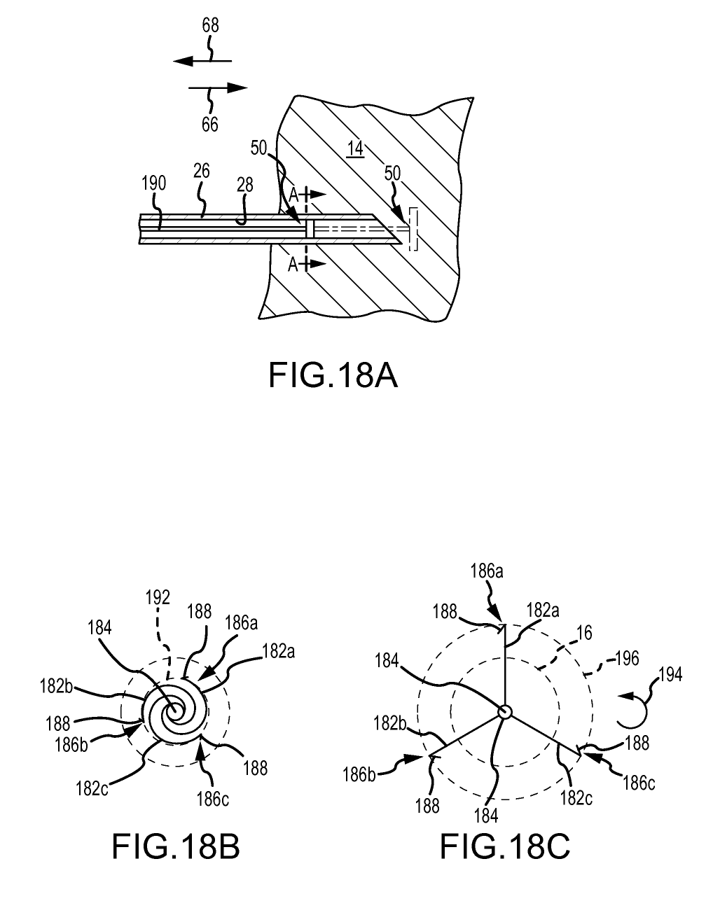

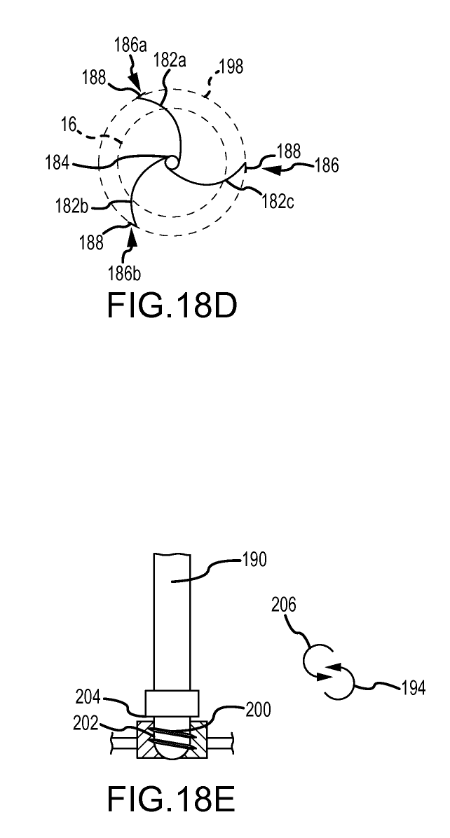

Referring to FIGS. 18A through 18E, in some embodiments, the reinforcing element 50 may be embodied as one or more spiral portions 182a-182c secured to a hub 184. Anchors 186a-186c may secure near distal ends of the spiral portions 182a-182c to engage the myocardium 14 and transfer a cinching load to the myocardium 14 in order to support ischemic area 16. In the illustrated embodiments, the anchors 186a-186c are embodied as a hooked portions 188 formed at the distal ends of the spiral portions 182a-182c. For example, the spiral portions 182a-182c may be formed of a resilient biocompatible polymer or metal. For example, the spiral portions 182a-182c may be embodied formed of nitinol wires and the anchors 186a-186c may be embodied as bent terminal portions of the nitinol wires forming the spiral portions 182a-182c. Although the anchors 186a-186c are shown as barbs in which the element reverses upon itself, it will be appreciated that any anchor configuration that resists motion through tissue more in one direction that another may be suitable for use as an anchor in accordance with this invention.

Referring specifically to FIG. 18A, in use the reinforcing element 50 may be positioned by inserting a needle 26 into the myocardium 14. The needle 26 may be inserted substantially perpendicularly to the outer surface of the myocardium 14 and may be located within the ischemic area 16. The reinforcing element 50 may then be forced in distal direction 66 out of the lumen 28 of the needle 26 into the ischemic area 16. The reinforcing element 50 may be forced out by means of a push rod 190 secured thereto and extending through the lumen 28 to a point located outside of a patient, or at least outside of the myocardium 14 in the case of an open chest procedure.

Referring to FIG. 18B, after exiting the needle 26, the spiral portions 182a-182c may be in a relaxed or compressed state illustrated having a first circumscribing diameter 192. The diameter 192 may be less than the diameter of the ischemic area 16. The push rod 190 may then be rotated in the direction 194 of spiraling of the spiral portions 182a-182c, causing the spiral portions 182a-182c to spread outwardly from the hub 184, as shown in FIG. 18C, to a second circumscribing diameter 196. The push rod 190 may then be detached from the hub 184 and the spiral portions 182a-182c may relax partially to the final orientation shown in FIG. 18D having a third circumscribing diameter 196. The third circumscribing diameter 196 may be less than the second diameter 196 and greater than the first diameter 192. Return of the spiral portions 182a-182c to a relaxed state may be inhibited by engagement of the anchors 186a-186c with the myocardium 14. In some embodiments, the spiral portions 182a-182c may be dimensioned such that the anchors 186a-186c are positioned within healthy tissue adjacent the ischemic area 16.

Referring to FIG. 18E, in some embodiments, the push rod 190 may be secured to the hub 184 by means of threads 200 secured to the push rod 190 engaging a threaded aperture 202 formed in the hub 184. A shoulder 204 secured to the push rod 190 may engage the hub 184 when the push rod 190 is rotated in the spiral direction 194 such that the push rod 190 forces the hub 184 to rotate rather than threading through the hub 184. To remove the push rod 190, it may be rotated in counter-spiral direction 206 to disengage the threads 200 from the threaded aperture 202.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus for supporting cardiac tissue including ischemic tissue comprising:
a first anchor configured to engage the cardiac tissue and resist movement therethrough;
a second anchor configured to engage the cardiac tissue and resist movement therethrough; and
a biasing member engaging the first and second anchors and configured to urge the first anchor toward the second anchor.

2. The apparatus of claim 1, wherein the first and second anchors comprise barbs coupled to opposing ends of the biasing member.

3. The apparatus of claim 1, wherein the biasing element comprises a first portion of a spring having a first outer diameter along a length thereof and wherein the first and second anchors comprise second and third portions of the spring located on opposite sides of the first portion and having a second and third outer diameters, respectively, the second and third outer diameter being greater than the first outer diameter.

4. The apparatus of claim 1, wherein the biasing member comprises a first arcuate member and a second arcuate member, a proximal end of the first arcuate member secured to a proximal end of the second arcuate member and the first anchor secured to a distal end of the first arcuate member and the second anchor secured to a distal end of the second arcuate member.

5. The apparatus of claim 4, wherein the biasing member further comprises a third arcuate member having a proximal end secured to the proximal ends of the first and second arcuate members and a distal end, the apparatus further comprising a third anchor secure to the distal end of the third arcuate member.

6. The apparatus of claim 5, wherein the first, second, and third arcuate members comprise spirals each spiraling in the same direction.

7. The apparatus of claim 4, wherein the first, second, and third arcuate members comprise elastic wires and wherein the first, second, and third anchors comprise bent distal portions of the elastic wires.

8. The apparatus of claim 1, wherein the biasing member comprises an elastic wire.

9. The apparatus of claim 8, wherein the elastic wire includes at least a portion forming at least one of an undulating pattern, a coil, and an arc.

10. The apparatus of claim 1, wherein the biasing element comprises an elastic material having a relaxed shape, the apparatus further comprising a bioabsorbable structure engaging the biasing element such that the biasing element is maintained in a deformed shape.

11. The apparatus of claim 1, wherein the biasing element comprises a material that undergoes a change in shape responsive to an electric field.

12. A method for treating cardiac tissue including ischemic tissue comprising:
positioning a catheter adjacent the ischemic tissue; and
urging a reinforcing member out of a lumen of the catheter into the ischemic tissue.

13. The method of claim 12, wherein the reinforcing member comprises a first anchor portion, a middle portion, and a second anchor portion and wherein urging a reinforcing member out of the lumen comprises:
urging the first anchor portion into the cardiac tissue;
urging the middle portion into the cardiac tissue while applying tension to the middle portion; and
urging the second anchor portion into the cardiac tissue while applying tension to the middle portion.

14. The method of claim 13, wherein the first and second anchor portions comprise barbs coupled to opposing ends of the biasing member.

15. The method of claim 13, wherein the middle portion comprises a first portion of a spring having a first width along a length thereof and wherein the first and second anchor portions comprise second and third portions of the spring located on opposite sides of the first portion and having a second and third widths, respectively, the second and third widths being greater than the first width.

16. The method of claim 13, further comprising disengaging a locking mechanism from the second anchor portion.

17. The method of claim 16, wherein the second anchor portion comprises an interference configured to engage the locking mechanism.

18. The method of claim 12, wherein the reinforcing element comprises a bioabsorbable member maintaining the reinforcing element in a deformed configuration.

19. The method of claim 12, wherein the reinforcing element comprises a shape memory material configured to assume a memory-shape following insertion into the cardiac tissue.

20. The method of claim 12, wherein urging the reinforcing member out of the lumen of the catheter into the ischemic tissue comprises:
urging a delivery needle out of the lumen of the catheter into the ischemic tissue; and
urging the reinforcing member out of a lumen of the delivery needle.

21. The method of claim 20, wherein urging the delivery needle out of the lumen of the catheter comprises bending the delivery needle.

22. The method of claim 21, wherein bending the delivery needle comprises urging the needle out of an aperture defined by a lateral surface of the catheter and in communication with the lumen of the catheter.

23. A method for supporting ischemic tissue within a myocardium comprising:
inserting a delivery needle within the myocardium;
urging a reinforcing element outwardly from the delivery needle into the myocardium, the reinforcing element comprising a plurality of spiral portions each secured to a hub and having an anchor portion; and
rotating the hub such that the spiral portions expand relative to the hub, the anchor portions of the spiral portions engaging the myocardium.

24. The method of claim 23, wherein rotating the hub comprises rotating a rod detachably secured to the hub and extending through the delivery needle.

25. The method of claim 23, wherein the spiral portions comprise elastic wires and wherein the anchor portions comprise bent distal portions of the elastic wires.

26. The method of claim 25, wherein the elastic wires comprise at least one of a biocompatible polymer and nitinol.
